(19) United States
(12) Patent Application Publication
(10) Pub. No.: US 2010/0221231 A1
(43) Pub. Date: Sep. 2, 2010

(54) Skin replacement compositions and methods

(76) Inventor: Charlotte A. Smith, Wexford, PA (US)

Correspondence Address: LINDA O. PALLADINO, 45 HONEYSUCKLE COURT, STORMVILLE, NY, 12582, US

(21) Appl. No.: 12/798,192

(22) Filed: Mar. 31, 2010

Related U.S. Application Data
(63) Continuation-in-part of application No. PCT/US08/11250, filed on Sep. 29, 2008.
(60) Provisional application No. 60/997,077, filed on Oct. 1, 2007.
(60) Provisional application No. 61/188,337, filed on Aug. 8, 2008.

(51) Int. Cl. A61K 35/12; C12N 5/071; A61P 17/02

(52) U.S. Cl. 424/93.7; 435/371

(57) ABSTRACT

The invention is directed to methods of making novel skin replacement compositions. The invention is further directed to the novel skin replacement compositions. The invention is also directed to methods for treating wounds, in particular burns and chronic, non-healing wounds, with such novel skin replacement compositions.

### CROSS-REFERENCE TO RELATED APPLICATIONS

This application is a Continuation-in-Part of International Application No. PCT/US2008/11250, filed Sep. 29, 2008, which claims priority under 35 USC §119(e) of U.S. Provisional Application Nos. 60/997,077, filed Oct. 1, 2007, and 61/188,337, filed Aug. 8, 2008, the entireties of which are incorporated herein by reference.

### FIELD OF THE INVENTION

The field of the invention is directed to methods of making novel skin replacement compositions. The field of the invention is further directed to the novel skin replacement compositions made by such method. The field of the invention is also directed to methods for treating wounds, in particular burns and chronic, non-healing wounds, with such novel skin replacement compositions.

### DESCRIPTION OF RELATED ART

Izumi, K., et al. (J Oral Maxillofac Surg, 1999, 57:571-577) describes the ex vivo development of a composite human oral mucosal equivalent. This oral mucosa equivalent is composed of oral mucosal keratinocytes and AlloDerm®. Izumi, K., et al. (J Dent Res, 2000, 79(3):798-805) describe the development and characterization of a tissue-engineered human oral mucosa equivalent produced in a serum-free culture system. This oral mucosa equivalent is composed of oral mucosal keratinocytes and AlloDerm®. Izumi, K., et al. (Tissue Eng, 2003, 9(1):163-174) describe the evaluation of transplanted tissue-engineered oral mucosa equivalents in severe combined immunodeficient mice. This composition is composed of human oral mucosal keratinocytes and AlloDerm®.

Yoshizawa, M., et al. (J Oral Maxillofac Surg, 2004, 62:980-988) describe ex vivo produced human conjunctiva and oral mucosa equivalents grown in s serum-free culture system. These compositions were composed of human conjunctival and oral mucosal keratinocytes, respectively, and AlloDerm®.

Song, J., et al. (J Biomed Mater Res A, 2004, 719(1):143-153) describe the development and characterization of a canine oral mucosa equivalent in a serum-free environment. This composition is composed of canine buccal mucosa keratinocytes and AlloDerm®.

Sun, T., et al (Tissue Eng 2005 11(11-12):1824-31) describe the development of a closed bioreactor system for culture of tissue-engineered skin at an air-liquid interface.

MatTek Corporation, 200 Homer Avenue, Ashland, Mass. 01721, manufacture a ready to use human tissue equivalent product called EpiDermFT™ which is made using normal human epidermal keratinocytes and normal human dermal fibroblasts. EpiDermFT™ is suitable for research use only. EpiDermFT™ is not suitable for use in humans.

### BACKGROUND OF THE INVENTION

The term “skin” is commonly used to describe the body covering of any animal, but technically it refers only to the body covering of vertebrates, who all have the same basic skin structure. The skin is essential to an organism's survival. It forms a physical barrier that helps prevent harmful microorganisms and chemicals from entering the body, prevents the loss of body fluids, protects internal body structures from injury, and protects from the potentially damaging ultraviolet rays of the sun. In addition, the skin helps regulate body temperature, excretes certain waste products, and is an important sensory organ. Skin contains various types of specialized nerve cells responsible for the sense of touch, pain, pressure, etc.

The skin is the body's largest organ. For example, the skin of an average adult male weighs 4.5 to 5 kg and measures about 2 sq m in area. It covers the surface of the body at a thickness of just 1.4 to 4.0 mm. The skin is thickest on areas of the body that are subjected to rubbing or friction such as the palms of the hands and the soles of the feet. Although quite delicate, the skin is very resilient, constantly regenerating itself and exhibiting a remarkable ability to repair itself after injury. The skin is made up of two distinct layers, the epidermis and the dermis. The epidermis is the outer layer of the skin and is a tough, waterproof, protective layer. The dermis, or inner layer, is thicker than the epidermis and gives the skin its strength and elasticity. The two layers of the skin are anchored to one another by a thin but complex layer of tissue known as the basement membrane which is composed of a series of elaborately interconnecting molecules that serve to hold the skin together. Below the dermis is the subcutaneous layer, the hypodermis, which is a layer of tissue composed of protein fibers and adipose tissue. Although not technically part of the skin itself, the subcutaneous layer contains glands and other skin structures, as well as sensory receptors involved in the sense of touch.

There is a high incidence of injury to the skin. One of the most serious types of injury is full-thickness wounds such as burns and chronic, non-healing ulcers (i.e. diabetic foot ulcers). A major problem facing clinicians treating severe burn injury is the limited availability of donor skin and the lack of success with currently available skin substitutes. Most of these treatments are suboptimal due to the likelihood of tissue rejection, the requirement for the donor skin or the skin substitute to be replaced numerous times during the healing process, and the disappointing functional and cosmetic outcomes generally obtained. Most of the available treatments that do manage to heal the wound form a skin that does not have any of the normal skin appendages such as hair or sweat or sebaceous glands, is usually discolored, and forms unsightly and deforming scars that often result in reduced mobility of the affected body part.

Clearly, this unmet medical need requires a treatment option that overcomes or at least minimizes these barriers to normal healing and functioning of skin.

### BRIERF SUMMARY OF THE INVENTION

It is, therefore, an object of the instant invention to provide novel skin replacement compositions and methods related thereto. Such compositions utilize novel cells including extraembryonic cytokine secreting cells (herein referred to as ECS cells) in combination with extracellular matrices. In a particular embodiment, the compositions utilize novel cells including, but not limited to, Amnion-derived Multipotent Progenitor cells (herein referred to as AMP cells), in combination with extracellular matrices, and further optionally in combination with conditioned media derived from ECS cells including AMP cells (such conditioned media derived from AMP cells is referred to herein as Amnion-derived Cellular Cytokine Solution or ACCS), cell lysates derived therefrom, and cell products derived therefrom.

A further object of the instant invention is a method of creating such novel skin replacement compositions. This novel method comprises seeding the ECS cells, including AMP cells, on top of a suitable matrix, for example AlloDerm® human dermis, and this cell/matrix (dermis) combination is incubated under conditions in which an air-liquid interface is achieved. In this embodiment, the matrix is placed in culture media such that the matrix is submerged in culture media but the cells (ECS cells, including AMP cells) are not submerged in media but rather are exposed on their apical surface to the humidified air in the incubator. This air-liquid interface arrangement recreates the environment in which skin cells are normally found during healthy regeneration, exposed to air on the outside and to a fluid medium on the inside. Novel skin replacement compositions thus created are suitable for use in the methods of the invention described herein.

It is also an object of the instant invention to promote accelerated full-thickness wound healing, particularly burn healing or chronic, non-healing wound (i.e. ulcer) healing, using the novel skin replacement compositions described herein. It is further an object of the invention to reduce or prevent scarring following wound healing and to promote the formation of stronger healed wounds by increasing tensile strength and/or breaking strength. It is also an object of the invention to promote the formation of skin appendages, for example, hair follicles, hair and/or sweat glands, using the novel skin replacement compositions.

Accordingly, a first aspect of the invention is a method of making a skin replacement composition comprising a) incubating ECS cells in keratinocyte differentiation media; b) seeding the ECS cells of step a) on an extracellular matrix; and c) incubating the ECS cells and the extracellular matrix of step b) under air-liquid interface conditions such that the ECS cells form a stratified epidermal layer on the extracellular matrix. In one embodiment, the ECS cells are AMP cells. In another embodiment the extracellular matrix is a natural matrix. In a specific embodiment the natural matrix is human dermis. In a very specific embodiment the human dermis is Alloderm® or F1exHD®. Still another embodiment is a skin replacement composition further comprising sweat glands and/or hair follicles and/or hair.

Aspect two of the invention is a method making a skin replacement composition comprising a) incubating AMP cells in keratinocyte differentiation media; b) seeding the AMP cells of step a) on Alloderm® human dermis; and c) incubating the AMP cells and the Alloderm® human dermis of step b) under air-liquid interface conditions such that the AMP cells form a stratified epidermal layer on the Alloderm® human dermis.

Aspect three of the invention is a skin replacement composition made by the methods of aspects one or two.

Aspect four of the invention is the use of a skin replacement composition comprising ECS cells and an extracellular matrix for promoting wound healing in a subject having a condition which would benefit therefrom. In one embodiment the wound is a burn. In another embodiment the wound is a chronic, non-healing wound. Such chronic, non-healing wounds include, for example, ulcers including but not limited to diabetic, pressure, venous and sickle cell ulcers.

Aspect five of the invention is a skin replacement composition comprising ECS cells and an extracellular matrix. In one embodiment the ECS cells are AMP cells. In a specific embodiment the AMP cells are cultured in basal medium which is supplemented with human serum or human serum albumin. In another embodiment the extracellular matrix is human dermis. In a specific embodiment the human dermis is Alloderm® or FlexHD®.

Aspect six of the invention is a method of making a skin replacement composition comprising a) incubating stem cells capable of differentiating into keratinocytes in keratinocyte differentiation media; b) seeding the stem cells of step a) on an extracellular matrix; and c) incubating the stem cells and the extracellular matrix of step b) under air-liquid interface conditions such that the stem cells form a stratified epidermal layer on the extracellular matrix.

Aspect seven of the invention is a skin replacement composition made by the method of aspect six.

Other aspects, features and advantages of the invention will be apparent from the accompanying description, examples and the claims. The contents of all references, pending patent applications and published patents, cited throughout this application are hereby expressly incorporated by reference. In case of conflict, the present specification, including definitions, will control.

### DEFINITIONS

As defined herein “isolated” refers to material removed from its original environment and is thus altered “by the hand of man” from its natural state.

As defined herein, a “gene” is the segment of DNA involved in producing a polypeptide chain; it includes regions preceding and following the coding region, as well as intervening sequences (introns) between individual coding segments (exons).

As used herein, the term “protein marker” means any protein molecule characteristic of the plasma membrane of a cell or in some cases of a specific cell type.

As used herein, “enriched” means to selectively concentrate or to increase the amount of one or more materials by elimination of the unwanted materials or selection and separation of desirable materials from a mixture (i.e. separate cells with specific cell markers from a heterogeneous cell population in which not all cells in the population express the marker).

As used herein, the term “substantially purified” means a population of cells substantially homogeneous for a particular marker or combination of markers. By substantially homogeneous is meant at least 90%, and preferably 95% homogeneous for a particular marker or combination of markers.

The term “placenta” as used herein means both preterm and term placenta.

As used herein, the term “totipotent cells” shall have the following meaning. In mammals, totipotent cells have the potential to become any cell type in the adult body; any cell type(s) of the extraembryonic membranes (e.g., placenta). Totipotent cells are the fertilized egg and approximately the first 4 cells produced by its cleavage.

As used herein, the term “pluripotent stem cells” shall have the following meaning. Pluripotent stem cells are true stem cells with the potential to make any differentiated cell in the body, but cannot contribute to making the components of the extraembryonic membranes which are derived from the trophoblast. The amnion develops from the epiblast, not the trophoblast. Three types of pluripotent stem cells have been confirmed to date: Embryonic Stem (ES) Cells (may also be totipotent in primates), Embryonic Germ (EG) Cells, and Embryonic Carcinoma (EC) Cells. These EC cells can be isolated from teratocarcinomas, a tumor that occasionally occurs in the gonad of a fetus. Unlike the other two, they are usually aneuploid.

As used herein, the term “multipotent stem cells” are true stem cells but can only differentiate into a limited number of types. For example, the bone marrow contains multipotent stem cells that give rise to all the cells of the blood but may not be able to differentiate into other cells types.

As used herein, the term “extraembryonic tissue” means tissue located outside the embryonic body which is involved with the embryo's protection, nutrition, waste removal, etc. Extraembryonic tissue is discarded at birth. Extraembryonic tissue includes but is not limited to the amnion, chorion (trophoblast and extraembryonic mesoderm including umbilical cord and vessels), yolk sac, allantois and amniotic fluid (including all components contained therein). Extraembryonic tissue and cells derived therefrom have the same genotype as the developing embryo.

As used herein, the term “extraembryonic cytokine secreting cells” or “ECS cells” means a population of cells derived from the extraembryonic tissue which have the characteristics of secreting a unique combination of physiologically relevant cytokines in a physiologically relevant temporal manner into the extracellular space or into surrounding culture media and which have not been cultured in the presence of any non-human animal materials, making them and cell products derived from them suitable for human clinical use. ECS cells may be selected from populations of cells and compositions described in this application and in US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179, the contents of which are incorporated herein by reference in their entirety.

As used herein, the term “amnion-derived multipotent progenitor cell” or “AMP cell” means a specific population of ECS cells that are epithelial cells derived from the amnion. In addition to the characteristics described above for ECS cells, AMP cells have the following characteristics. They have not been cultured in the presence of any non-human animal materials, making them and cell products derived from them suitable for human clinical use. In a particular and specific embodiment, the AMP cells are cultured in basal media supplemented with human serum or human serum albumin. In an another particular and specific embodiment, the AMP cells secrete the cytokines VEGF, Angiogenin, PDGF and TGFβ2 and the MMP inhibitors TIMP-1 and/or TIMP-2. The physiological range of the cytokine or cytokines in the unique combination is as follows: ˜5-16 ng/mL for VEGF, ˜3.5-4.5 ng/mL for Angiogenin, ˜100-165 pg/mL for PDGF, ˜2.5-2.7 ng/mL for TGFβ2, ˜0.68 μg mL for TIMP-1 and ˜1.04 μg/mL for TIMP-2. The AMP cells may optionally express Thymosin β4. AMP cells grow without feeder layers, do not express the protein telomerase and are non-tumorigenic. AMP cells do not express the hematopoietic stem cell marker CD34 protein. The absence of CD34 positive cells in this population indicates the isolates are not contaminated with hematopoietic stem cells such as umbilical cord blood or embryonic fibroblasts. Virtually 100% of the cells react with antibodies to low molecular weight cytokeratins, confirming their epithelial nature. Freshly isolated amnion-derived cells, from which AMP cells are isolated, will not react with antibodies to the stein/progenitor cell markers c-kit (CD117) and Thy-1 (CD90). Several procedures used to obtain cells from full term or pre-term placenta are known in the art (see, for example, US 2004/0110287; Anker et al., 2005, Stem Cells 22:1338-1345; Ramkumar et al., 1995, Am. J. Ob. Gyn. 172:493-500). However, the methods used herein provide improved compositions and populations of cells. AMP cells have previously been described as “amnion-derived cells” (see U.S. Provisional Application Nos. 60/666,949, 60/699,257, 60/742,067, U.S. Provisional Application Nos. 60/813,759, U.S. application Ser. No. 11/333,849, U.S. application Ser. No. 11/392,892, and PCTUS06/011392, each of which is incorporated herein in its entirety).

By the term “animal-free” when referring to certain compositions, growth conditions, culture media, etc. described herein, is meant that no non-human animal-derived materials, such as bovine serum, proteins, lipids, carbohydrates, nucleic acids, vitamins, etc., are used in the preparation, growth, culturing, expansion, storage or formulation of the certain composition or process. By “no non-human animal-derived materials” is meant that the materials have never been in or in contact with a non-human animal body or substance so they are not xeno-contaminated. Only clinical grade materials, such as recombinantly produced human proteins, are used in the preparation, growth, culturing, expansion, storage and/or formulation of such compositions and/or processes.

By the term “serum-free” when referring to certain compositions, growth conditions, culture media, etc. described herein, is meant that no animal-derived serum (i.e. no non-human) is used in the preparation, growth, culturing, expansion, storage or formulation of the certain composition or process.

By the term “expanded”, in reference to cell compositions, means that the cell population constitutes a significantly higher yield of cells than is obtained using previous methods. For example, the level of cells per gram of amniotic tissue in expanded compositions of AMP cells is at least 50 and up to 150 fold higher than the number of cells in the primary culture after 5 passages, as compared to about a 20 fold increase in such cells using previous methods. In another example, the level of cells per gram of amniotic tissue in expanded compositions of AMP cells is at least 30 and up to 100 fold higher than the number of cells in the primary culture after 3 passages. Accordingly, an “expanded” population has at least a 2 fold, and up to a 10 fold, improvement in cell numbers per gram of amniotic tissue over previous methods. The term “expanded” is meant to cover only those situations in which a person has intervened to elevate the number of the cells.

As used herein, the term “passage” means a cell culture technique in which cells growing in culture that have attained confluence or are close to confluence in a tissue culture vessel are removed from the vessel, diluted with fresh culture media (i.e. diluted 1:5) and placed into a new tissue culture vessel to allow for their continued growth and viability. For example, cells isolated from the amnion are referred to as primary cells. Such cells are expanded in culture by being grown in the growth medium described herein. When such primary cells are subcultured, each round of subculturing is referred to as a passage. As used herein, “primary culture” means the freshly isolated cell population.

As used herein, “conditioned medium” is a medium in which a specific cell or population of cells has been cultured, and then removed. When cells are cultured in a medium, they may secrete cellular factors that can provide support to or affect the behavior of other cells. Such factors include, but are not limited to hormones, cytokines, extracellular matrix (ECM), proteins, vesicles, antibodies, chemokines, receptors, inhibitors and granules. The medium containing the cellular factors is the conditioned medium. Examples of methods of preparing conditioned media are described in U.S. Pat. No. 6,372,494 which is incorporated by reference in its entirety herein. As used herein, conditioned medium also refers to components, such as proteins, that are recovered and/or purified from conditioned medium or from ECS cells, including AMP cells.

As used herein, the term “Amnion-derived Cellular Cytokine Solution” or “ACCS” means conditioned medium that has been derived from AMP cells or expanded AMP cells. In a particular and specific embodiment, the AMP cells have been cultured in basal media supplemented with human serum or human serum albumin. Amnion-derived cellular cytokine solution or ACCS has previously been referred to as “amnion-derived cytokine suspension”.

The term “physiological level” as used herein means the level that a substance in a living system is found and that is relevant to the proper functioning of a biochemical and/or biological process.

As used herein, the term “pooled” means a plurality of compositions that have been combined to create a new composition having more constant or consistent characteristics as compared to the non-pooled compositions. For example, pooled AMP cells have more constant or consistent characteristics compared to non-pooled AMP cells.

The term “therapeutically effective amount” means that amount of a therapeutic agent necessary to achieve a desired physiological effect (i.e. accelerated wound healing).

The term “lysate” as used herein refers to the composition obtained when cells, for example, AMP cells, are lysed and optionally the cellular debris (e.g., cellular membranes) is removed. Lysis may be achieved by mechanical means, by freezing and thawing, by sonication, by use of detergents, such as EDTA, or by enzymatic digestion using, for example, hyaluronidase, dispase, proteases, and nucleases.

As used herein, the term “substrate” means a defined coating on a surface that cells attach to, grown on, and/or migrate on. As used herein, the term “matrix” means a substance that cells grow in or on that may or may not be defined in its components. The matrix includes both biological and non-biological substances. As used herein, the term “scaffold” means a three-dimensional (3D) structure (substrate and/or matrix) that cells grow in or on. It may be composed of biological components, synthetic components or a combination of both. Further, it may be naturally constructed by cells or artificially constructed. In addition, the scaffold may contain components that have biological activity under appropriate conditions.

As used herein, the term “pharmaceutically acceptable” means that the components, in addition to the therapeutic agent, comprising the formulation, are suitable for administration to the patient being treated in accordance with the present invention.

As used herein, the term “tissue” refers to an aggregation of similarly specialized cells united in the performance of a particular function.

As used herein, the term “therapeutic protein” includes a wide range of biologically active proteins including, but not limited to, growth factors, enzymes, hormones, cytokines, inhibitors of cytokines, blood clotting factors, peptide growth and differentiation factors.

The term “transplantation” as used herein refers to the administration of a composition comprising cells that are either in an undifferentiated, partially differentiated, or fully differentiated form, or a combination thereof, into a human or other animal.

As used herein, the terms “a” or “an” means one or more; at least one.

As used herein, the term “adjunctive” means jointly, together with, in addition to, in conjunction with, and the like.

As used herein, the term “co-administer” can include simultaneous or sequential administration of two or more agents.

“Treatment,” “treat,” or “treating,” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition, i.e., arresting its development; (c) relieving and or ameliorating the disease or condition, i.e., causing regression of the disease or condition; or (d) curing the disease or condition, i.e., stopping its development or progression. The population of subjects treated by the methods of the invention includes subjects suffering from the undesirable condition or disease, as well as subjects at risk for development of the condition or disease.

As used herein, a “wound” is any disruption, from whatever cause, of normal anatomy (internal and/or external anatomy) including but not limited to traumatic injuries such as mechanical (i.e. contusion, penetrating), thermal, chemical, electrical, radiation, concussive and incisional injuries; elective injuries such as operative surgery and resultant incisional hernias, fistulas, etc.; acute wounds, chronic wounds, infected wounds, and sterile wounds, as well as wounds associated with disease states (i.e. ulcers caused by diabetic neuropathy or ulcers of the gastrointestinal or genitourinary tract). A wound is dynamic and the process of healing is a continuum requiring a series of integrated and interrelated cellular processes that begin at the time of wounding and proceed beyond initial wound closure through arrival at a stable scar. These cellular processes are mediated or modulated by humoral substances including but not limited to cytokines, lymphokines, growth factors, and hormones. In accordance with the subject invention, “wound healing” refers to improving, by some form of intervention, the natural cellular processes and humoral substances of tissue repair such that healing is faster, and/or the resulting healed area has less scaring and/or the wounded area possesses tissue strength that is closer to that of uninjured tissue and/or the wounded tissue attains some degree of functional recovery.

As used herein, the term “skin substitute” means a composition that is used to dress a wound. Skin substitutes may be natural or synthetic. Such skin substitutes are general temporary because they rarely implant into the recipient's tissue and are typically rejected by the recipient when made with allogeneic cells.

As used herein, the term “skin replacement” means a composition that is designed to replace both the dermal and the epidermal components of skin and that can incorporate into the recipient's skin without rejection. The novel compositions described herein are skin replacement compositions.

As used herein, the term “skin appendages” means the eccrine and apocrine sweat glands, hair follicles, sebaceous glands, and nails. Except for nails, all the skin appendages are located in the dermis.

### DETAILED DESCRIPTION

In accordance with the present invention there may be employed conventional molecular biology, microbiology, and recombinant DNA techniques within the skill of the art. Such techniques are explained fully in the literature. See, e.g., Sambrook et al, 2001, “Molecular Cloning: A Laboratory Manual”; Ausubel, ed., 1994, “Current Protocols in Molecular Biology” Volumes I-III; Celis, ed., 1994, “Cell Biology: A Laboratory Handbook” Volumes I-III; Coligan, ed., 1994, “Current Protocols in Immunology” Volumes I-III; Gait ed., 1984, “Oligonucleotide Synthesis”; Hames & Higgins eds., 1985, “Nucleic Acid Hybridization”; Hames & Higgins, eds., 1984, “Transcription And Translation”; Freshney, ed., 1986, “Animal Cell Culture”; IRL Press, 1986, “Immobilized Cells And Enzymes”; Perbal, 1984, “A Practical Guide To Molecular Cloning.”

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

### Therapeutic Uses

Wound healing—The instant invention is based upon the discovery that novel skin replacement compositions comprising undifferentiated, partially differentiated or fully differentiated ECS cells, and in particular, AMP cells, including combinations thereof, in combination with extracellular matrices, in particular human dermis such as AlloDerm®, and optionally in combination with other agents such as ECS cell conditioned media, including ACCS, cell lysates derived from ECS cells including AMP cells, cell products derived from ECS cells including AMP cells, and extracellular matrices derived from ECS cells including AMP cells, can accelerate the wound healing process for all wound types, but especially for full-thickness wounds such as burns and chronic, non-healing ulcers (i.e. diabetic foot ulcers). Such wound healing occurs when the skin replacement compositions are administered topically, i.e. to the surface of the wound site. Using skin replacement compositions the wounds undergo healing more rapidly than similar wounds left to heal naturally or which are treated with currently available methods i.e. skin substitutes.

The compositions and methods of the present invention are effective in accelerating healing of wounds caused by a number of sources, including but not limited to incisional, compression, thermal, radiation, acute, chronic, infected, sterile and congenital injuries.

In addition to accelerating wound healing, the skin replacement compositions of the invention prevent and/or reduce the incidence of wound failure by increasing both breaking strength and tensile strength of wounds as well as increasing the rate in which increased breaking strength and tensile strength is attained during the wound healing process. Thus, wounds not only heal faster, but become stronger faster than wounds treated with other available agents or untreated.

The skin replacement compositions of the invention are applied in a therapeutically effective amount to accomplish accelerated wound healing, including increased wound strength and decreased wound failure. A “therapeutically effective amount” of a therapeutic agent within the meaning of the present invention will be determined by a patient's attending physician or veterinarian. Such amounts are readily ascertained by one of ordinary skill in the art and will enable accelerated wound healing when administered in accordance with the present invention. Factors which influence what a therapeutically effective amount will be include, the specific activity of the therapeutic agent being used, the wound type (mechanical or thermal, full or partial thickness, etc.), the size of the wound, the wound's depth (if full thickness), the absence or presence of infection, time elapsed since the injury's infliction, and the age, physical condition, existence of other disease states, and nutritional status of the patient. Additionally, other medication the patient may be receiving will effect the determination of the therapeutically effective amount of the therapeutic agent to administer.

In a preferred embodiment of the present invention, skin replacement compositions of the invention should be topically administered to the wound site to promote accelerated wound healing in the patient. This topical administration can be as a single application of the skin replacement composition or as repeated applications given at multiple designated intervals. A single application of the skin replacement composition is preferable. It will readily be appreciated by those skilled in the art that the preferred application regimen will vary with the type, size and severity of the injury being treated.

Formulations suitable for topical administration in accordance with the present invention comprise therapeutically effective amounts of the skin replacement compositions with one or more pharmaceutically acceptable carriers and/or adjuvants. Skin replacement compositions may be used in conjunction with a variety of materials routinely used in the treatment of wounds, such as collagen based creams, films, microcapsules, or powders; hyaluronic acid or other glycosaminoglycan-derived preparations; creams, foams, suture material; and routine wound dressings.

Reconstructive and cosmetic surgery—The compositions and methods of the present invention are effective in accelerating healing following reconstructive and cosmetic surgery. It's estimated that more that one million reconstructive procedures are performed by surgeons every year. The goals of reconstructive surgery differ from those of cosmetic surgery. Reconstructive surgery is performed on abnormal structures of the body, caused by birth defects, developmental abnormalities, trauma or injury, infection, tumors, or disease. It is generally performed to improve function, but may also be done to approximate a normal appearance. Cosmetic surgery is performed to reshape normal structures of the body to improve the patient's appearance and self-esteem (i.e. rhinoplasty).

### Differentiation of ECS Cells, Including AMP Cells, and Differentiated Cell Types

The ECS cells, including AMP cells, may be contacted with various growth factors (termed differentiation factors) that influence differentiation of such stem cells into particular cell types such as skin cells, muscle cells, bone cells and nerve cells. Skilled artisans will recognize that other cell types, including various types of stem cells, which are capable of differentiating into keratinocytes may be suitable for use in practicing the methods of the invention.

The literature is replete with differentiation protocols for embryonic as well as non-embryonic stem or other multipotent cells, including stem cells (see for example osteogenic differentiation (Shi, Y. Y., et al., (2005) Plast Reconstr Surg 116, 1686-96.); adipogenic differentiation (Shi, Y. Y., et al., (2005) Plast Reconstr Surg 116, 1686-96.); chondrogenic differentiation (Malladi, P., et al., (2006) Am J Physiol Cell Physiol 290, C1139-46.); keratinocyte differentiation using commercially available differentiation media (Celprogen Human Keratinocyte Stem Cell Complete Differentiation Media catalog # M3600-09D; Cascade Biologics Epi-life catalog # M-Epicf-500). One skilled in the art will recognize that any of these protocols or media may be applied to the ECS cell compositions, including the AMP cell compositions described herein, to produce partially or fully differentiated cells for use in preparing the skin replacement compositions of the invention.

Differentiated cells derived from ECS cells including AMP cells may be detected and/or enriched by the detection of tissue-specific markers by immunological techniques, such as flow immunocytochemistry for cell-surface markers, immunohistochemistry (for example, of fixed cells or tissue sections) for intracellular or cell-surface markers, Western blot analysis of cellular extracts, and enzyme-linked immunoassay, for cellular extracts or products secreted into the medium. The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods. Keratinocyte and/or epithelial cell makers such as involucrin, filaggrin and cytokeratin are examples of markers which may be detected in the skin replacement compositions of the invention.

Alternatively, differentiated cells may be detected using selection markers. For example, AMP cells can be stably transfected with a marker that is under the control of a tissue-specific regulatory region as an example, such that during differentiation, the marker is selectively expressed in the specific cells, thereby allowing selection of the specific cells relative to the cells that do not express the marker. The marker can be, e.g., a cell surface protein or other detectable marker, or a marker that can make cells resistant to conditions in which they die in the absence of the marker, such as an antibiotic resistance gene (see e.g., in U.S. Pat. No. 6,015,671).

### Isolation, Identification and Characterization of ECS Cells Including AMP Cells

Various methods for isolating cells from the extraembryonic tissue, which may then be used to produce the ECS cells of the instant invention are described in the art (see, for example, US2003/0235563, US2004/0161419, US2005/0124003, U.S. Provisional Application Nos. 60/666,949, 60/699,257, 60/742,067, 60/813,759, U.S. application Ser. No. 11/333,849, U.S. application Ser. No. 11/392,892, PCTUS06/011392, US2006/0078993, PCT/US00/40052, U.S. Pat. No. 7,045,148, US2004/0048372, and US2003/0032179).

Identifying ECS cells—Once extraembryonic tissue is isolated, it is necessary to identify which cells in the tissue have the characteristics associated with ECS cells (see definition above). For example, cells are assayed for their ability to secrete a unique combination of cytokines into the extracellular space or into surrounding culture media.

AMP cell compositions are prepared using the steps of a) recovery of the amnion from the placenta, b) dissociation of the cells from the amniotic membrane, c) culturing of the dissociated cells in a basal medium, for example IMDM, with the addition of a naturally derived and/or recombinantly produced human protein, for example, human serum or human serum albumin; d) selecting the adherent cells (the AMP cells) and discarding the non-adherent cells from the cell culture, and optionally e) further proliferation of the cells, optionally using additional additives and/or growth factors (i.e. recombinant human EGF). Details are contained in US Publication No. 2006-0222634-A1, which is incorporated herein by reference.

Culturing of the AMP cells—The cells are cultured in a basal medium. Such medium includes, but is not limited to, EPILIFE® culture medium for epithelial cells (Cascade Biologicals), OPTI-PRO™ serum-free culture medium, VP-SFM serum-free medium, IMDM highly enriched basal medium, KNOCKOUT™ DMEM low osmolality medium, 293 SFM II defined serum-free medium (all made by Gibco; Invitrogen), HPGM hematopoietic progenitor growth medium, Pro 293S-CDM serum-free medium, Pro 293A-CDM serum-free medium, U1traMDCKTM serum-free medium (all made by Cambrex), STEMLINE® T-cell expansion medium and STEMLINE® II hematopoietic stem cell expansion medium (both made by Sigma-Aldrich), DMEM culture medium, DMEM/F-12 nutrient mixture growth medium (both made by Gibco), Ham's F-12 nutrient mixture growth medium, M199 basal culture medium (both made by Sigma-Aldrich), and other comparable basal media. Such media should either contain human protein or be supplemented with human protein. As used herein a “human protein” is one that is produced naturally or one that is produced using recombinant technology. “Human protein” also is meant to include a human fluid or derivative or preparation thereof, such as human serum, human serum albumin, or amniotic fluid, which contains human protein. In a specific embodiment, the basal media is IMDM highly enriched basal medium. In another embodiment the basal medium is STEMLINE® T-cell expansion medium or STEMLINE® II hematopoietic stem cell expansion medium, or OPTI-PRO™ serum-free culture medium, or combinations thereof and the human protein is human albumin at a concentration of at least 0.5% and up to 10%. In particular embodiments, the human albumin concentration is from about 0.5 to about 2%. The human albumin may come from a liquid or a dried (powder) form and includes, but is not limited to, recombinant human albumin, PLASBUMIN® normal human serum albumin and PLASMANATE® human blood fraction (both made by Talecris Biotherapeutics).

In a most preferred embodiment, the cells are cultured using a system that is free of animal products to avoid xeno-contamination. In this embodiment, the culture medium is IMDM highly enriched basal medium, STEMLINE® T-cell expansion medium or STEMLINE® II hematopoietic stem cell expansion medium, OPTI-PRO™ serum-free culture medium, or DMEM culture medium, with human serum albumin (for example PLASBUMIN® normal human serum albumin) added up to concentrations of 10%. The invention further contemplates the use of any of the above basal media wherein animal-derived proteins are replaced with recombinant human proteins and animal-derived serum, such as BSA, is replaced with human albumin. In preferred embodiments, the media is serum-free in addition to being animal-free.

Optionally, other factors are used. In one embodiment, epidermal growth factor (EGF) at a concentration of between 0.1 μg/mL is used. In a preferred embodiment, the EGF concentration is around 10 ng/mL. Alternative growth factors which may be used include, but are not limited to, TGFα or TGFβ2 (5 ng/mL; range 0.1-100 ng/mL), activin A, cholera toxin (preferably at a level of about 0.1 μg/mL; range 0-10 μg/mL), transferrin (5 μg/mL; range 0.1-100 μg/mL), fibroblast growth factors (bFGF 40 ng/mL (range 0-200 ng/mL), aFGF, FGF-4, FGF-8; (all in range 0-200 ng/mL), bone morphogenic proteins (i.e. BMP-4) or other growth factors known to enhance cell proliferation. All supplements are clinical grade.

In a specific embodiment, the following method is used to obtain selected AMP cells. The cells are plated into plastic tissue culture vessels (i.e. T75 flasks) immediately upon isolation from the amnion. After ˜1-5 days, preferably ˜1-3 days, and most preferably ˜2 days in culture, non-adherent cells are removed from the plastic tissue culture vessel and discarded and the adherent cells are kept. This attachment of cells to a plastic tissue culture vessel is the selection method used to obtain the desired population of AMP cells. Adherent and non-adherent AMP cells appear to have similar cell surface marker expression profiles but the adherent cells have the advantage of possessing greater viability than the non-adherent population of cells and are thus the desired population of AMP cells. Adherent AMP cells are cultured until they reach ˜13,000-700,000 cells/cm², preferably ˜53,000-500,000 cells/cm² and most preferably ˜120,000-300,000 cells/cm². At this point, the cultures are confluent or close to confluent. Suitable cells cultures will reach this number of cells between ˜5-14 days, preferably between 5-9 days. Attaining this criterion is an indicator of the proliferative potential of the AMP cells and cells that do not achieve this criterion are not selected for further analysis and use. Once the AMP cells reach ˜13,000-700,000 cells/cm², preferably ˜53,000-500,000 cells/cm² and most preferably ˜120,000-300,000 cells/cm², they are removed from the plastic tissue culture vessel and cryopreserved. This collection time point is called p0.

The AMP cells of the invention are characterized by assaying for physiologically relevant cytokines. Suitable cells are those in which each cytokine occurs in the physiological range of ˜5.0-16 ng/mL for VEGF, ˜3.5-4.5 ng/mL for Angiogenin, ˜100-165 pg/mL for PDGF, ˜2.5-2.7 ng/mL for TGFβ2, ˜0.68 μg/mL for TIMP-1 and ˜1.04 μg/mL for TIMP-2. The cells may optionally be assayed for Thymosin β4.

In addition, the AMP cells of the invention are further characterized as follows: Using commercially available antibodies to known stem cell markers, freshly isolated AMP cells have been extensively characterized. Briefly, freshly isolated amnion epithelial cells are substantially negative with respect to CD90 and CD 117. In addition, such cell populations are essentially negative for protein expression of CD34, CD44, CD45, CD140b, CD105; essentially positive for protein expression of CD9 and CD29; between about 70-95% positive for protein expression of SSEA4, CD10, CD166 and CD227; between about 60-95% positive for protein expression of HLA-G, EGFR and CD26; and between about 10-50% positive for protein expression of CD71. Details on this procedure are contained in US Publication No. 2006-0222634-A1, which is incorporated herein by reference.

In alternative embodiments substantially purified AMP cell populations can be created using antibodies against protein markers expressed (positive selection) or not expressed (negative selection) on the cell surface of the AMP cells. These antibodies may be used to identify, characterize, isolate or create such substantially purified populations of AMP cells expressing those protein markers using a variety of methods. Details on this procedure are contained in U.S. Publication No. 2006-0222634-A1, which is incorporated herein by reference.

In addition, protein markers that are not expressed on the surface of AMP cells may also be used to identify, isolate or create populations of AMP cells not expressing those markers. Such procedures may involve a negative selection method, such as passage of sample cells over a column containing anti-protein marker antibodies or by binding of cells to magnetic bead-conjugated antibodies to the protein markers or by panning on plates coated with protein marker antibodies and collecting the unbound cells. Alternatively, a single-cell suspension may be exposed to one or more fluorescent-labeled antibodies that immuno-specifically bind to the protein markers. Details on this procedure are contained in US Publication No. 2006-0222634-A1, which is incorporated herein by reference.

Generation of ECS conditioned medium- is obtained as described below for ACCS, except that ECS cells are used.

Generation of ACCS—The AMP cells of the invention can be used to generate ACCS. In one embodiment, the AMP cells are isolated as described herein and 1>10⁶ cells/mL are seeded into T75 flasks containing between 5-30 mL culture medium, preferably between 10-25 mL culture medium, and most preferably about 10 mL culture medium. In a specific and preferred embodiment, the culture medium is preferably a basal medium (for example IMDM highly enriched basal medium) which is supplemented with human serum or human serum albumin. The cells are cultured until confluent, the medium is changed and in one embodiment the ACCS is collected 1 day post-confluence. In another embodiment the medium is changed and ACCS is collected 2 days post-confluence. In another embodiment the medium is changed and ACCS is collected 4 days post-confluence. In another embodiment the medium is changed and ACCS is collected 5 days post-confluence. In a preferred embodiment the medium is changed and ACCS is collected 3 days post-confluence. In another preferred embodiment the medium is changed and ACCS is collected 3, 4, 5, 6 or more days post-confluence. Skilled artisans will recognize that other embodiments for collecting ACCS from AMP cell cultures, such as using other tissue culture vessels, including but not limited to cell factories, flasks, hollow fibers, or suspension culture apparatus, or collecting ACCS from sub-confluent and/or actively proliferating cultures, are also contemplated by the methods of the invention. It is also contemplated by the instant invention that the ACCS be cryopreserved following collection. It is also contemplated by the invention that ACCS be lyophilized following collection. It is also contemplated by the invention that ACCS be formulated for sustained-release following collection. It is also contemplated that ACCS production be scaled up for generation of sufficient product for clinical testing and for commercialization. Skilled artisans are familiar with cryopreservation lyophilization, and sustained-release formulation methodologies.

### Compositions

The present invention provides pharmaceutical compositions of skin replacement compositions, and a pharmaceutically acceptable carrier. The term “pharmaceutically acceptable” means approved by a regulatory agency of the Federal or a state government or listed in the U.S. Pharmacopeia or other generally recognized pharmacopeia for use in animals, and more particularly, in humans. The term “carrier” refers to a diluent, adjuvant, excipient, or vehicle with which the composition is administered. Such pharmaceutical carriers can be sterile liquids, such as water and oils, including those of petroleum, animal, vegetable or synthetic origin, such as peanut oil, soybean oil, mineral oil, sesame oil and the like. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Examples of suitable pharmaceutical carriers are described in “Remington's Pharmaceutical Sciences” by E. W. Martin, and still others are familiar to skilled artisans.

### Generation of Skin Replacement Compositions

To generate skin replacement compositions, ECS cells, including AMP cells, are cultured in combination with matrices under air-liquid interface conditions. Preferred matrices in which the ECS cells, including AMP cells, can be used in combination with include the commercially available extracellular matrix products such as Oasis®, Dermagraft®, DressSkin®, AlloDerm®, Promogran®, EZ-Derm®, etc. For example, the ECS cells, including AMP cells, can be placed on top of these products and then applied to the wound to accelerate wound healing. Such application may occur immediately upon combining the products or after incubation for a period of time. Other suitable extracellular matrices familiar to skilled artisans are contemplated by the methods of the invention.

In a particular embodiment, the ECS cells, including AMP cells, are seeded on top of AlloDerm® and this cell/matrix (dermis) combination is incubated under conditions in which an air-liquid interface is achieved. In this embodiment, the AlloDerm® is placed in culture media such that the AlloDerm® is submerged in culture media but the cells (ECS cells, including AMP cells) are not submerged in media but rather are exposed on their apical surface to the humidified air in the incubator. This air-liquid interface arrangement recreates the environment in which skin cells are normally found during healthy regeneration, exposed to air in the outside and to a fluid medium on the inside. Skin replacement compositions thus created are suitable for use in the methods of the invention described herein. In one embodiment, the AMP cells that are seeded on the matrix are p0cells. In other embodiments, p1, p2, p3, etc., AMPs cells are seeded on the matrix. In other embodiments, other cell types, including various stem cell types, which are capable of differentiating into keratinocytes may be suitable for use in practicing the methods of the invention.

Such compositions are especially useful in treating wounds such as burns and non-healing wounds (i.e. diabetic ulcers, pressure ulcers, sickle cell ulcers, venous ulcers, etc.). Importantly, ECS cells, including the AMP cells, have been shown to be non-immunogenic (they do not express HLA-G upon isolation and they do not express Class II antigens at any time (see U.S. Application Publication No. 2006-0222634 and PCT US08/00396, each incorporated by reference herein). Therefore, the skin replacement composition is not likely to be rejected and very likely to become incorporated into the patient's tissue, becoming a permanent part of the patient's skin until normal skin regeneration completely replaces it.

In certain embodiments, the wound is primed with ACCS prior to application of the skin replacement composition to the wound. In another embodiment, ACCS is applied to the wound after the skin replacement composition is applied. In another embodiment, the skin replacement composition is soaked (i.e. rehydrated) in ACCS prior to placement on the wound. In other embodiments, ACCS is applied prior to and after the skin replacement composition is applied to the wound. The exact protocol for application of the skin replacement composition, optionally in combination with ACCS, will need to be determined by the attending physician when the particular wound is evaluated.

It is also contemplated by the methods of the invention that the skin replacement compositions be stored in ACCS (either full strength or partial strength) prior to use.

### Formulation, Dosage and Administration

The skin replacement compositions of the invention may be administered to a subject to provide various cellular or tissue functions, for example, to accelerate wound healing. As used herein “subject” may mean either a human or non-human animal.

One of skill in the art may readily determine the appropriate dose or application of skin replacement compositions for a particular purpose. The skilled artisan will recognize that a preferred dose or application is one which produces a therapeutic effect, such as accelerated wound healing, in a patient in need thereof. Determination of a proper dose or application of skin replacement compositions will require empirical determination at time of use based on several variables including but not limited to the severity and type of wound being treated; patient age, weight, sex, health; other medications and treatments being administered to the patient; and the like. One of skill in the art will also recognize that number of doses or applications (application regimen) to be administered needs also to be empirically determined based on, for example, severity and type of wound being treated. In a preferred embodiment, one dose is sufficient to accelerated wound healing. Other embodiments contemplate, 2, 3, 4, or more doses to accelerate wound healing, etc.

Skilled artisans will recognize that any and all of the standard methods and modalities for wound healing currently in clinical practice are suitable for practicing the methods of the invention. Routes of administration, formulation, co-administration with other agents (if appropriate) and the like are discussed in detail elsewhere herein.

Timing of treatment and administration of the compositions of the invention is also dependent upon the severity and type of wound being treated. For example, in the case of traumatic wounds wherein treatment is only possible after the injury has occurred, it may be advantageous to treat the wound immediately upon presentation and again following medical and/or surgical intervention. Attending physicians will determine the exact treatment regimen based on the severity of the wound being treated, etc. In the case of chronic wounds, treatment should be initiated as soon as possible for best possible outcome.

It may be desirable to administer the skin replacement compositions in combination with other agents, including active agents and/or inactive agents. Active agents include but are not limited to ECS cell conditioned media, including ACCS, growth factors, cytokines, chemokines, antibodies, antibiotics, anti-fungals, anti-virals, other cell types, and the like. Inactive agents include carriers, diluents, stabilizers, gelling agents, delivery vehicles, ECMs (natural and synthetic), scaffolds, and the like.

ECS cell conditioned media including ACCS may also be inserted into a delivery device, e.g., a syringe, in different forms, for use in combination treatments.

### Treatment Kits

The invention also provides for an article of manufacture comprising packaging material and a pharmaceutical composition of the invention contained within the packaging material, wherein the pharmaceutical composition comprises a skin replacement composition. In preferred embodiments, the skin replacement composition comprises substantially purified populations of cells, for example AMP cells, in combination with a human dermis such as AlloDerm®. The packaging material comprises a label or package insert which indicates that the skin replacement composition can be used for treating a variety of disorders including but not limited to burns and non-healing ulcers (i.e. full-thickness burns and non-healing diabetic, pressure, venous, and sickle cell ulcers).

### EXAMPLES

The following examples are put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

### Example 1

### Preparation of AMP Cell Compositions

Recovery of AMP cells—AMP cells were dissociated from starting amniotic membrane using the dissociation agents PXXIII. The number of cells recovered per g of amnion was about 10-15×10⁶ for dissociation with PXXIII.

Method of obtaining selected AMP cells—Cells were plated immediately upon isolation from the amnion. After ˜2 days in culture non-adherent cells were removed and the adherent cells were kept. This attachment to a plastic tissue culture vessel is the selection method used to obtain the desired population of AMP cells. Adherent and non-adherent AMP cells appear to have a similar cell surface marker expression profile but the adherent cells have greater viability and are the desired population of cells. Adherent AMP cells were cultured in IMDM highly enriched basal medium supplemented with 2% human serum or human serum albumin until they reached ˜120,000-150,000 cells/cm². At this point, the cultures were confluent. Suitable cell cultures will reach this number of cells between ˜5-14 days. Attaining this criterion is an indicator of the proliferative potential of the AMP cells and cells that do not achieve this criterion are not selected for further analysis and use. Once the AMP cells reached ˜120,000-150,000 cells/cm², they were collected and cryopreserved. This collection time point is called p0.

### Example 2

### Generation of ACCS

The AMP cells of the invention can be used to generate ACCS, including pooled ACCS. The

AMP cells were isolated as described above and ˜1×10⁶ cells/mL were seeded into T75 flasks containing ˜10 mL culture medium as described above. The cells were cultured until confluent, the medium was changed and ACCS was collected 3 days post-confluence. Skilled artisans will recognize that other embodiments for collecting ACCS from confluent cultures, such as using other tissue culture vessels, including but not limited to cell factories, flasks, hollow fibers, or suspension culture apparatus, etc. are also contemplated by the methods of the invention (see Detailed Description above). It is also contemplated by the instant invention that the ACCS be cryopreserved, lyophilized or formulated for sustained-release following collection. It is also contemplated that ACCS be collected at different time point (see Detailed Description for details).

### Example 3

### Generation of Pooled ACCS

ACCS was obtained essentially as described above. In certain embodiments, ACCS was collected multiple times from an AMP cell culture derived from one placenta and these multiple ACCS collections were pooled together. Such pools are referred to as “SP pools” (more than one ACCS collection/one placenta). In another embodiment, AMP cell cultures were derived from several placentas, i.e. from 5 or 10 placentas. The AMP cells from each placenta were cultured and one ACCS collection from each culture was collected and then they were all pooled. These pools are termed “MP1 pools” (one ACCS collection/placenta, multiple placentas). In yet another embodiment, AMP cell cultures were derived from several placentas, i.e. from 5 or 10 placentas. The AMP cells from each placenta were cultured and more than one ACCS collection was performed from each AMP cell culture and then pooled. These pools are termed “MP2 pools” (more than one ACCS collection/placenta, multiple placentas).

### Example 4

### Generation of Skin Replacement Compositions

The keratinocyte differentiation markers involucrin and filaggrin are not expressed by AMP cells that have been seeded onto a matrix. To determine whether or not culturing the AMP cells in keratinocyte differentiation media (i.e. Epi-life (Cascade Biologics M-EPIcf-500) or Celprogen prior to seeding onto a matrix (i.e. Alloderm®, LifeCell B 18493) can stimulate up-regulation of cytokeratin, involucrin, or filaggrin, t0 AMP cells were seeded at 12×10⁶/T-75 flask and incubated at 37° C. in 10 mLs of either IMDM media, Epi-life media, or Celprogen media until they reached confluency (p0 cells). The confluent AMP cells were trypsinized, counted and 250 μL AMP cell solution seeded onto the Alloderm® matrix at 1×10⁶ cells/mL.

Switching to an air-liquid interface (ALI) environment—All samples were submerged for the first 4 days. On day 5, half of the samples are switched to an air-liquid interface using Transwell plates as follows. The Alloderm®/AMP cell samples were transferred into Transwell plates and 500 μL of IMDM media was added to the lower compartment only, leaving the topmost layer of the samples exposed to the air.

Results—Basement membrane proteins (collagen IV, collagen VII, and laminin) are present in all conditions and are maintained throughout 28 days. Collagen VII staining is faint on some air-liquid interface samples. The AMP cells seem to detach from the basement membrane by the 28 day time point. FITC staining for involucrin, filaggrin and cytokeratin is visible in all samples by day 14.The air-liquid interface samples have better keratin expression than the submerged samples and the Epi-Life air-liquid interface samples seem to have better keratin expression than the IMDM air-liquid interface samples.

### Example 5

### Evaluation of Skin Replacement Compositions in a Standard Animal Model

The skin replacement compositions described in Example 5 are evaluated in a standard animal model of wound healing. Briefly, the methods are: The skin replacement compositions are tested in vivo against the ECM alone as a control. Half of the control grafts have ACCS applied onto the excisional bed prior to grafting. When grafting the skin replacement compositions into rat excisional wounds, the grafts are sutured with monofilament nylon. The grafts are covered with bridal veil, then a layer of Adaptic, and then a cellulose foam stent. This is sutured or stapled into place to prevent dislodgement. The sutures are removed on day seven when the first biopsies are taken. Two rats are sacrificed each 7 days for 21 days. The healed grafts are sectioned such that they include the underlying rat wound so that the interface can also be studied. The sections are preserved for frozen sectioning, for permanent section histology and for scanning EM. Among other tests, the sections are stained for H&E, trichrome, picrosirius and Verhoef's. In addition, the rats are evaluated for accelerated healing, incorporation of skin replacement compositions into existing tissue, lack of rejection and formation of skin appendages.

The present invention may be embodied in other specific forms without departing from the spirit or essential attributes thereof. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims.

Throughout the specification various publications have been referred to. It is intended that each publication be incorporated by reference in its entirety into this specification.

What is claimed is,:

1. A method of making a skin replacement composition comprising
a) incubating extraembryonic cytokine secreting (ECS) cells in keratinocyte differentiation media;
b) seeding the ECS cells of step a) on an extracellular matrix; and
c) incubating the ECS cells and the extracellular matrix of step b) under air-liquid interface conditions such that the ECS cells form a stratified epidermal layer on the extracellular matrix.

2. The method of claim 1 wherein the ECS cells are Amnion-derived Multipotent Progenitor (AMP) cells.

3. The method of claim 2 wherein the AMP cells are cultured in basal medium supplemented with human serum or human serum albumin.

4. The method of claim 1 wherein the extracellular matrix is a natural matrix.

5. The method of claim 4 wherein the natural matrix is human dermis.

6. The method of claim 5 wherein the human dermis is Alloderm® or FlexHD®.

7. A method making a skin replacement composition comprising
a) incubating AMP cells in keratinocyte differentiation media;
b) seeding the AMP cells of step a) on Alloderm®; and
c) incubating the AMP cells and the Alloderm® of step b) under air-liquid interface conditions such that the AMP cells form an epidermal layer on the Alloderm®.

8. The method of claim 1 or 7 wherein the skin replacement composition further comprises sweat glands and/or hair follicles and/or hair.

9. A skin replacement composition made by the methods of claim 1 or 7.

10. A method for promoting wound healing in a patient in need thereof comprising administering the skin replacement composition of claim 9.

11. The method of claim 10 wherein the wound is a burn.

12. The method of claim 10 wherein the wound is a chronic, non-healing wound.

13. A skin replacement composition comprising ECS cells and an extracellular matrix.

14. The skin replacement composition of claim 13, wherein the ECS cells are AMP cells.

15. The skin replacement composition of claim 14, wherein the extracellular matrix is human dermis.

16. The skin replacement composition of claim 15 wherein the human dermis is Alloderm® or FlexHD®.

17. A method of making a skin replacement composition comprising
a) incubating stem cells capable of differentiating into keratinocytes in keratinocyte differentiation media;
b) seeding the stem cells of step a) on an extracellular matrix; and
c) incubating the stem cells and the extracellular matrix of step b) under air-liquid interface conditions such that the stem cells form an epidermal layer on the extracellular matrix.

18. A skin replacement composition made by the method of claim 17.
